(19) United States
(12) Patent Application Publication
(10) Pub. No.: US 2003/0085714 A1
(43) Pub. Date: May 8, 2003

(54) Mass flow control in a process gas analyzer

(76) Inventors: Marion A. Keyes, St. Louis, MO (US); Stephen T. Staphanos, Long Beach, CA (US)

Correspondence Address: Christopher R. Christenson, WESTMAN CHAMPLIN & KELLY, International Centre, Suite 1600, 900 South Second Avenue, Minneapolis, MN, 55402-3319, US

(21) Appl. No.: 10/013,013

(22) Filed: Nov. 5, 2001

(51) Int. Cl. G01N 27/62

(52) U.S. Cl. 324/464

(57) ABSTRACT

A sample conditioner system carries a real-time sample of a process gas through a chromatograph column in an analyzer. A flame ionization detector (FID) is coupled to the chromatograph column and generates a temperature output and an output indicating sample ions. A processor generates a real-time process gas analysis and a mass set point. A flow controller controls mass flow of a stream of gas to the FID.

### FIELD OF THE INVENTION

The present invention relates to process gas analyzers. In particular, the present invention relates to process gas chromatographs.

### BACKGROUND OF THE INVENTION

Process gas analyzers are installed in a chemical process plant environment and connected to a control system to provide real-time data for use in control of the process plant. Process gas analyzers run unattended and are installed near a sample point rather than in a laboratory. Process gas analyzers are typically enclosed in a special housing to provide compatibility with the hazardous plant environment.

With process gas analyzers, conditions of the sampled process gas, such as pressure, temperature and chemical concentration can vary over time. Also, pressures and temperature of other gases supplied to the gas analyzer such as carrier gas supply, combustion air supply, or combustible gas supply can vary over time. Variations in temperature and pressure can have an adverse effect on the operating point of a flame ionization detector (FID) which detects various chemical species in the process gas. Because the process gas analyzer is installed in the field, however, there is no technician or operator continuously attending the process gas chromatograph to make corrective adjustments to bring the flame ionization detector back to an optimal operating point.

The process gas analyzer includes a chromatograph column and analyzes multiple chemical components of the process gas. When the rate of elution from the column is set at a relatively slow rate to provide adequate separation of a difficult to resolve pair of chemical species, then the time needed to elute other chemical species becomes excessive in relation to the time requirement for real-time output to the process control system. The real-time ability of the analyzer output is thus degraded for some applications.

A process gas analyzer is needed that has real-time speed in a wider variety of applications and also improved ability to adjust for variations in process or supply gas conditions in real time.

### SUMMARY OF THE INVENTION

Disclosed is a process gas analyzer for analyzing a process gas. The analyzer includes a sample conditioner system carrying a real-time sample of the process gas to a chromatograph column. The analyzer also includes a flame ionization detector (FID) that is coupled to the chromatograph column for receiving the real-time sample. The flame ionization detector generates a temperature output and an output indicating sample ions in the real-time sample.

A processor in the analyzer includes a process control system interface that generates a real-time process gas analysis as a function of the output indicating sample ions. The processor also generates a first set point for mass flow as a function of the temperature output. A flow controller in the analyzer passes a first stream of gas to the flame ionization detector. The flow controller includes a mass flow sensor providing a first sensor output. The flow controller further includes a valve regulating the mass flow of the first stream of gas as a function of the first set point and the first sensor output.

These and various other features as well as advantages which characterize the present invention will be apparent upon reading of the following detailed description and review of the associated drawings.

### BRIEF DESCRIPTION OF THE DRAWINGS

FIG. 1 illustrates a PRIOR ART arrangement of a process gas analyzer.

FIG. 2 illustrates a PRIOR ART arrangement of a flame ionization detector (FID).

FIGS. 3-4 together schematically illustrate a process gas analyzer.

FIG. 5 schematically illustrates a mass flow controller in a process gas analyzer.

FIG. 6 illustrates a cycle time of a PRIOR ART arrangement of a process gas analyzer.

FIG. 7 illustrates a reduced cycle time of an improved process gas analyzer.

### DETAILED DESCRIPTION OF THE PREFERRED EMBODIMENTS

A process gas analyzer is disclosed that includes one or more mass flow controllers that control flow of one or more gases to a flame ionization detector. A temperature sensor in the flame ionization detector provides feedback to a processor. The processor calculates a set point for the mass flow controller as a function of the sensed temperature. Improved control over the flow of gas to the flame ionization detector is achieved. The improved control can be used to control gas flows during ignition of the flame ionization detector. The improved control can also be used to control gas flows during process gas analysis.

In one preferred arrangement, the processor maintains a set point for mass flow at a relatively constant level over a first time interval and then increases the set point substantially linearly over a second time interval. This arrangement reduces the cycle time for analyzing a process gas with multiple components and improves the real-time performance of the process gas analyzer.

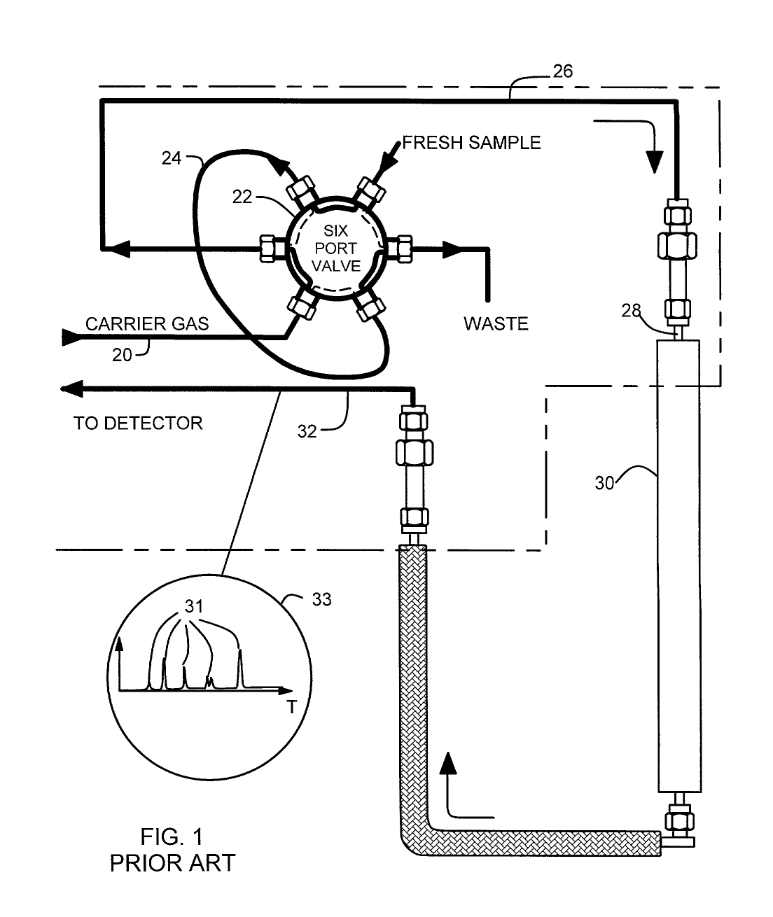

As illustrated in FIG. 1, a solvent (“carrier”) gas supplied at line 20 to a six port valve 22 transports a mixture of unknown chemicals from a sample loop 24 along a line 26 to a chromatograph column 28 that includes a heater 30. The column 28 includes a tube with a chemically adsorbent material that is packed in the tube or coated on the inside of the tube. Each unknown chemical moves through the column 28 at a different rate depending on its interaction with the solvent and the adsorbent material. Each chemical flows out of the column 28 at a different time. The chemicals flowing out of the column are transported along line 32 as a series of peaks 31 of chemical concentrations illustrated at 33. The peaks are separated in time, and each peak represents a different chemical compound.

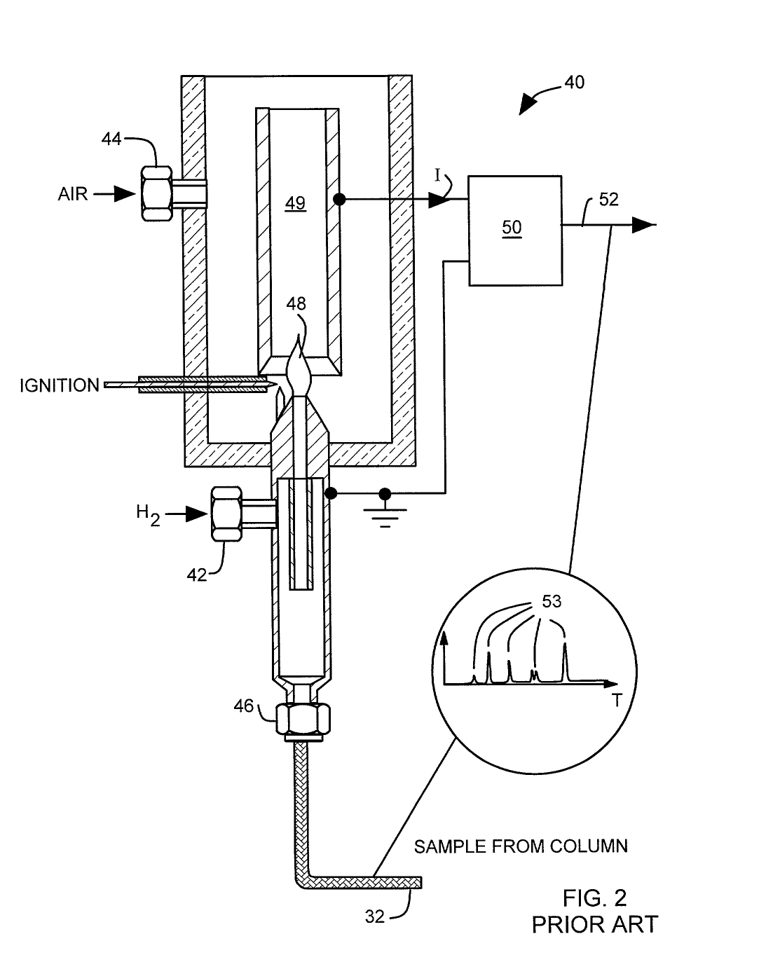

As illustrated in FIG. 2, line 32 (from FIG. 1) couples to a flame ionization detector (FID) 40 which provides high sensitivity and a wide dynamic range of detection. Individual chemical compounds are identified by the time that the individual peaks exit the column 28 (FIG. 1) and the peaks are quantified by the flame ionization detector (FID) 40. Flame ionization detector 40 receives a supply of combustible gas (hydrogen) at inlet 42, a supply of air at inlet 44 and a supply of the chemicals flowing out of the column 28 at inlet 46. The chemicals flowing out of the column 28 are ionized in a flame 48. An electronic circuit 50 senses an electrical current “I” passing through ionized gases 49 above the flame 48 and provides an electrical output 52 that represents the electrical current. The electrical output 52 has peaks 53 corresponding to the chemical species detected.

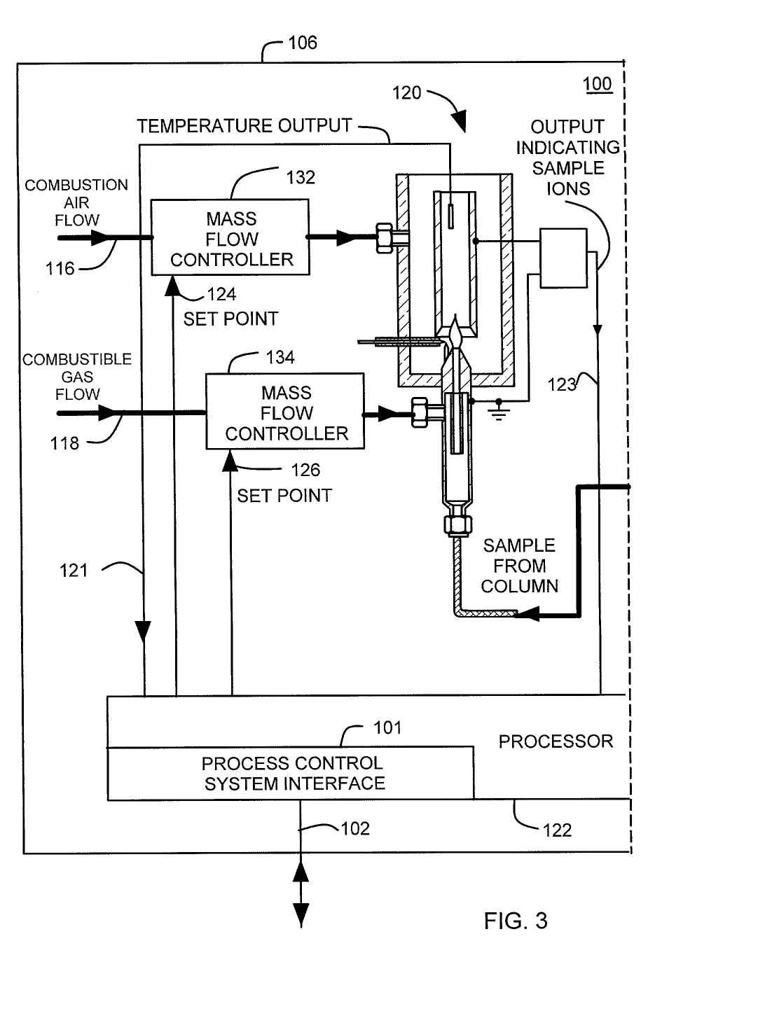

FIGS. 3-4 together schematically illustrate an improved process gas analyzer 100. FIGS. 3-4 can be best understood when joined along the dashed lines to form a single schematic. The process gas analyzer 100 is specially adapted for installation in a chemical process plant environment and is connectable to a control system to provide real-time data on line 102 for use in control of the process plant. Process gas analyzer 100 can run unattended and is installed near a sample point 103 to allow a real-time sample 104 to flow through the analyzer 100. Process gas analyzer 100 is preferably enclosed in a special housing 106 to provide compatibility with the hazardous plant environment. In process gas analyzer 100, a “front end” or sample conditioning system (SCS) 108 is coupled between a sample point 103 in the process plant and a six port valve 110. The sample conditioning system 108 is customized for the particular chemical plant application where it is installed, and typically includes a pressure reduction regulator, a filter and a flow controller that ensure that the sample reaching a chromatograph column 112 is a real-time sample that is properly conditioned for chromatography. The sample conditioning system 108 may be partially or fully included in enclosure 106. A portion of the sample conditioning system 108 can be constructed outside the process gas chromatograph enclosure 106, depending on the needs of the application.

Conditions of the sampled process at sample point 103, such as pressure and temperature can vary over time. Pressures and temperatures of gases supplied to the process gas analyzer such as carrier gas supply 114, combustion air supply 116, or combustible gas supply 118 can vary over time. In improved process gas analyzer 100, these pressure and temperature variations have substantially no effect on the operating point of a flame ionization detector (FID) 120. There is no need for a technician or operator to continuously attend the process gas analyzer 100 to make corrective adjustments to bring the flame ionization detector 120 back to an optimal operating point. As explained in more detail below, process gas analyzer 100 includes a processor 122 providing one or more set point outputs 124, 126, 128, 130 respectively to one or more mass flow controllers 132, 134, 136, 138 that control mass flow of gases that ultimately reach the flame ionization detector 120. The processor 122 and the mass flow controllers 132, 134, 136, 138 provide real-time control or adjustment of gas flows. The operating point of the flame ionization detector 120 remains stable, and continuous attendance by a technician is not needed.

Process gas analyzer 100 analyzes a process gas in a sample flow 104. The sample conditioner system 108 carries a real-time sample 104 of the process gas through a chromatograph column 112 in the process gas analyzer 100. The flame ionization detector (FID) 120 is coupled to the chromatograph column 112. The flame ionization detector 120 receives the real-time sample and generates a temperature output 121 and also an output 123 indicating sample ions in the real-time sample 104.

The processor 122 includes a process control system interface 101 that generates a real-time process gas analysis output 102 as a function of the output 123 indicating sample ions. Process control system interface 101 preferably produces output 102 as a telemetry output (formatted as Hart, Foundation Fieldbus, Profibus, or other known field bus protocol or a wireless signal) which can be sent to a control room. Typically, the process analyzer (including processor 122) is located remotely from the control system.

The processor 122 also generates at least one set point 124, 126, 128 or 130 for mass flow as a function of the temperature output 123. At least one flow controller 132, 134, 136 or 138 passes a stream of gas to the flame ionization detector 120 that is controlled based on the temperature output 123. The selection of the number and placement of mass flow controllers used depends on the needs of the application.

Processor 122 provides a controlled heating current 140 to column 112 and receives a column temperature signal 142 from the column 112. Processor 122 also provides a control signal 144 that controls actuation of the six port valve 110.

In operation, improved analyzer 100 can be configured to closely regulate or control the flow of one or more gases which have variations in pressure or flow that are a problem in a particular application. Mass flow controller 132 can be controlled by set point 124 to control the mass flow of combustion air from combustion air supply 116 to the flame ionization detector 120. Mass flow controller 134 can be controlled by set point 126 to control the mass flow of combustible gas from combustible gas supply 118 to the flame ionization detector. Mass flow controller 136 can be controlled by set point 128 to control the mass flow of the process gas sample 104 to the six port valve 110. Mass flow controller 138 can be controlled by set point 130 to control the mass flow of chemicals eluted from column 112 to the flame ionization detector 120. Processor 122 provides the mass flow set points 124, 126, 128 or 130 based on temperature sensed in the flame ionization detector. In a preferred embodiment, one or more mass flows are controlled to provide a substantially constant sensed temperature in the flame ionization detector during chemical analysis. Additionally, during a purge cycle of six port valve 110, set point 130 to mass flow controller 138 can be set to a high mass flow rate to provide rapid cooling of column 112 in preparation for a subsequent analysis cycle.

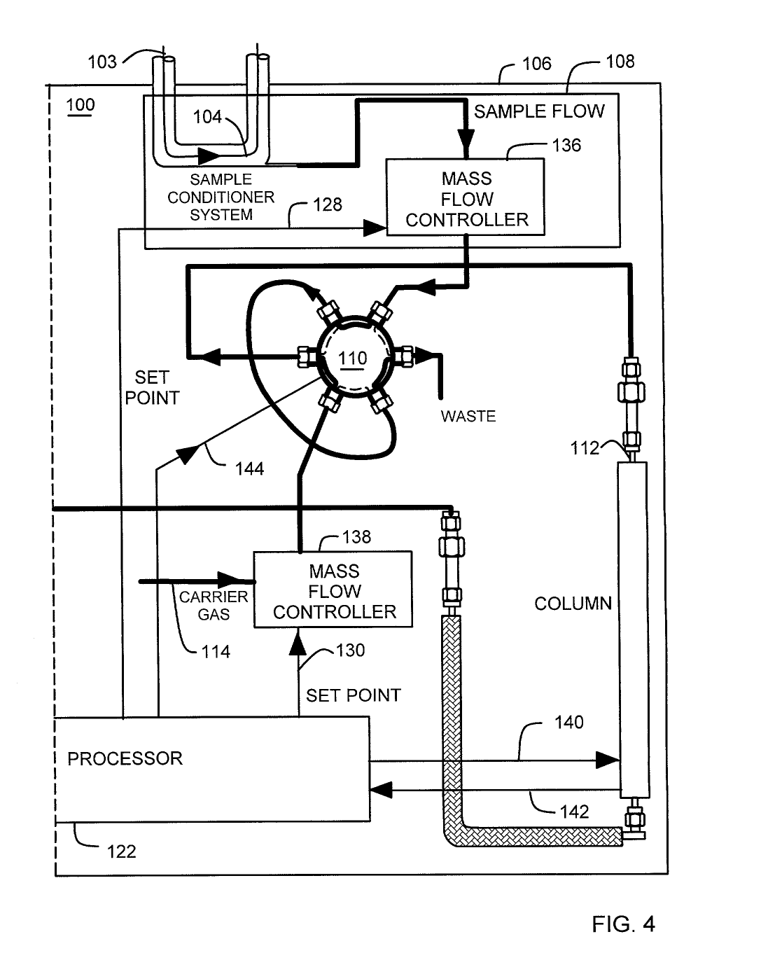

As illustrated in FIG. 5, a mass flow controller 200 is an example of one or more of the mass flow controllers 132, 134, 136 or 138 in FIGS. 3-4. Mass flow controller 200 includes a thermal mass flow sensor 202 providing a sensor output 204. The flow controller 200 further includes a valve 206 regulating the mass flow of a stream of gas 208 as a function of a mass flow set point 210 (corresponding with mass flow set points 124, 126, 128, 130 in FIGS. 3-4) and the sensor output 204. Mass flow controller 200 includes an electronic circuit 212 that compares the mass flow sensor output 204 to the mass flow set point 210 and generates an error signal 214. Error signal 214 is amplified and conditioned by a control circuit 216 to provide a control output 218 for the valve 206. Control circuit 216 can perform proportional, integral and/or differential control functions as needed to provide a stable mass flow.

As illustrated in FIG. 5, the mass flow set point 210 is generated by the processor 122 (shown also in FIGS. 3-4). Processor 122 compares the temperature output 121 of the flame ionization detector 120 to a temperature set point 230 and generates a temperature error output 232. Temperature error output 232 is amplified and conditioned by circuit 234 to provide the mass flow set point 210. Control circuit 234 can perform proportional, integral and/or differential control functions as needed to provide a stable mass flow set point 210. The temperature set point 230 can be a fixed value stored in memory of processor 122, or a calculated value calculated by processor 122, or a time varying signal generated by processor 122 for varying elution rate during an analysis cycle.

While only a single mass flow controller 200 is illustrated in FIG. 5, it will be understood that two or more mass flow controllers can be included in the process gas analyzer as illustrated in FIGS. 3-4. The stream of gas flow that is controlled by the mass flow controller 200 can be the sample of the process gas, the carrier gas, the combustion air flow or the combustible gas flow. In one preferred arrangement, the processor 122 controls a ratio of combustion air mass flow to combustible gas mass flow during ignition of the FID 120. In another preferred arrangement, the processor controls a ratio of combustion air mass flow to combustible gas mass flow to the FID 120 during process gas analysis. The processor 122 adjusts the sensitivity of the FID by simultaneously adjusting the mass flows of multiple streams of gas.

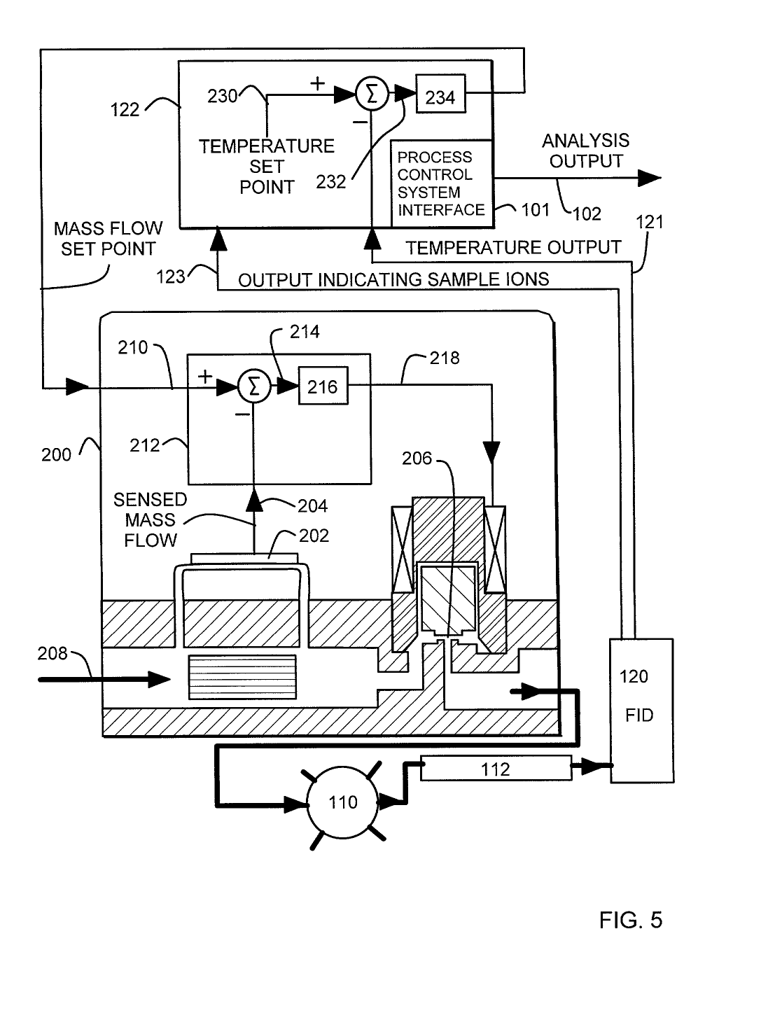

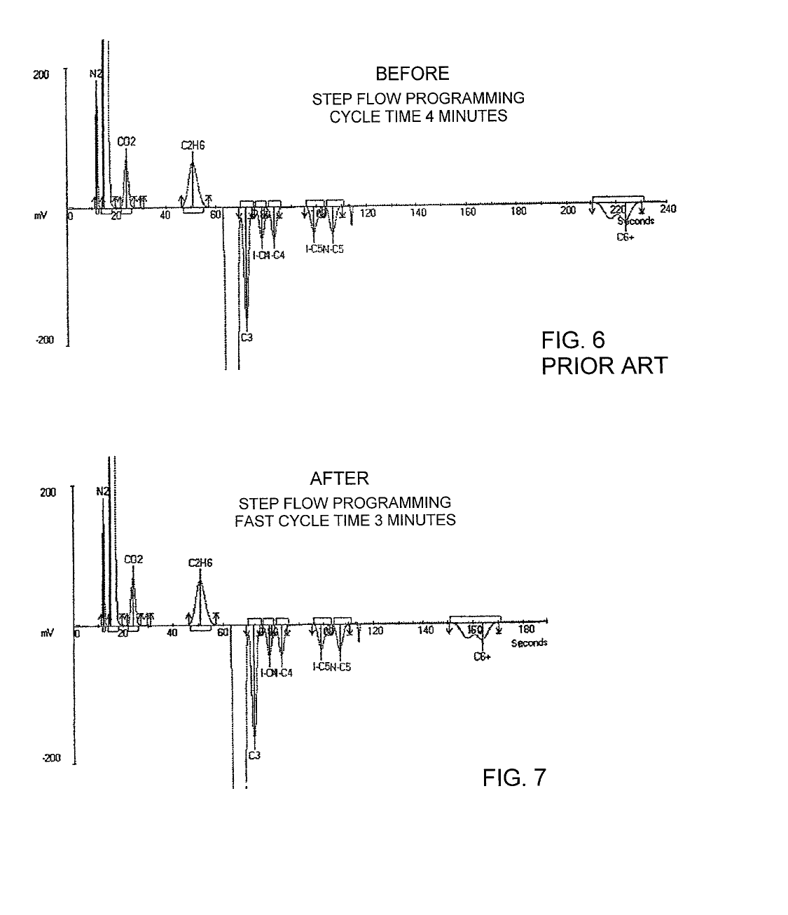

During an analysis cycle in one application, the processor 122 maintains the mass flow set point 130 for the carrier gas at a substantially constant level over a first time interval and then increases the mass flow set point 130 substantially linearly over a second time interval. This arrangement provides a relatively slow rate of elution to provide adequate separation of a difficult to resolve pair of chemical species, then the flow rate increases linearly to provide rapid identification of species that elute at much later times. The total analysis time is reduced and the real time requirements of output 102 can be met for many applications that were difficult in the past. As illustrated in FIG. 6, a “before” chemical analysis with a fixed elution rate took a cycle time of approximately 240 seconds to complete. After the programmed mass flow rates are used as illustrated in FIG. 7, the analysis cycle time is reduced to approximately 180 seconds. In FIG. 7 there is a substantially constant lower flow rate of carrier gas for 120 seconds, and then flow is increased linearly after 120 seconds until the slowest chemical species is detected.

Although the present invention has been described with reference to preferred embodiments, workers skilled in the art will recognize that changes may be made in form and detail without departing from the spirit and scope of the invention.

What is claimed is:

1. A process gas analyzer for analyzing a process gas, comprising:
a chromatograph column;
a sample conditioner system carrying a real-time sample of the process gas to the chromatograph column;
a flame ionization detector (FID) coupled to the chromatograph column for receiving the real-time sample and generating a temperature output and an output indicating sample ions in the real-time sample;
a processor including a process control system interface generating a real-time process gas analysis as a function of the output indicating sample ions, the processor also generating a first set point for mass flow as a function of the temperature output; and
a flow controller passing a first stream of gas to the flame ionization detector, the flow controller including a mass flow sensor providing a first sensor output, the flow controller further including a valve regulating the mass flow of the first stream of gas as a function of the first set point and the first sensor output.

2. The process gas analyzer of claim 1 wherein the first stream of gas is the sample of the process gas.

3. The process gas analyzer of claim 1 wherein the first stream of gas is a carrier gas.

4. The process gas analyzer of claim 1 wherein the first stream of gas is a combustion air flow.

5. The process gas analyzer of claim 1 wherein the first stream of gas is a combustible gas flow.

6. The process gas analyzer of claim 1 wherein the processor controls a ratio of combustion air mass flow to combustible gas mass flow during ignition of the FID.

7. The process gas analyzer of claim 1 wherein the processor controls a ratio of combustion air mass flow to combustible gas mass flow to the FID during process gas analysis.

8. The process gas analyzer of claim 1 wherein the first stream of gas is a carrier gas and wherein the processor maintains the first set point at a substantially constant level over a first time interval and then increases the first set point substantially linearly over a second time interval.

9. The process gas analyzer of claim 1 wherein the processor generates a second set point, the process gas analyzer further comprising:
a second flow controller passing a second stream of gas to the flame ionization detector, the second flow controller including a second mass flow sensor providing a second sensor output, the second flow controller further including a second valve regulating the mass flow of the second stream of gas as a function of the second set point and the second sensor output.

10. The process gas analyzer of claim 9 wherein the processor adjusts the sensitivity of the FID by adjusting the mass flows of the first and second streams of gas.

11. The process gas analyzer of claim 9 wherein the processor adjusting the mass flows of the first and second streams of gas to a substantially constant set point during a first time interval and to a substantially linearly increasing set point during a second time interval.

12. A method of analyzing a process gas, comprising:
passing a real-time sample of the process gas through a chromatograph column in a process gas analyzer;
generating a temperature output and an output indicating sample ions in the real-time sample in a flame ionization detector (FID) coupled to the chromatograph for receiving the real-time sample;
generating a real-time process gas analysis at a process control interface as a function of the output indicating sample ions;
generating a first set point for mass flow as a function of the temperature output;
passing a first stream of gas to the flame ionization detector through a flow controller;
generating a first sensor output from a first mass flow sensor in the flow controller; and
regulating the mass flow of the first stream of gas with a valve as a function of the first set point and the first sensor output.

13. A process gas analyzer for analyzing a process gas, comprising:
a chromatograph column;
a sample conditioner system carrying a real-time sample of the process gas to the chromatograph column;
a flame ionization detector (FID) coupled to the chromatograph column for receiving a real-time sample and generating a temperature output and an output indicating sample ions in the real-time sample;
a process control system interface generating a real-time process gas analysis as a function of the output indicating sample ions; and
means for sensing and controlling mass flow of a first stream of gas flowing to the flame ionization detector as a function of the temperature output, the sensed mass flow and a mass flow set point.
